(19) United States
(12) Patent Application Publication
(10) Pub. No.: US 2007/0083128 A1
(43) Pub. Date: Apr. 12, 2007

(54) Adaptive real-time line noise suppression for electrical or magnetic physiological signals

(76) Inventors: Wayne Cote, El Paso, TX (US); Curtis W. Ponton, El Paso, TX (US)

Correspondence Address: FULBRIGHT & JAWORSKI L.L.P., 80 SOUTH EIGHTH STREET, SUITE 2100, MINNEAPOLIS, MN, 55402, US

(21) Appl. No.: 11/546,543

(22) Filed: Oct. 10, 2006

(30) Foreign Application Priority Data
Oct. 10, 2005 (AU) 2005905546

(51) Int. Cl. A61B 5/04

(52) U.S. Cl. 600/544; 600/546

(57) ABSTRACT

The present invention provides a method of overcoming the contamination of physiological signals with noise caused by characteristics of the electrical supply to measuring devices. The method exploits the periodic and spectrally stationary nature of noise. The method can be implemented in software for easy calculation and display of calculated results for interpretation and use of the resulting relatively uncontaminated signals. The method can be applied where measurements are made of physiological parameters of humans or any other animal. The invention includes apparatus for acquiring and processing physiological signals from a subject included at least one sensor for acquiring at least one signal and at least one microprocessor means for processing the at least one signal, the microprocessor means including means for storing a whole number multiple of an artefact waveform for calculating the line-noise component of data derived from the at least one sensor.

### FIELD OF THE INVENTION

This invention relates to methods for analysis of outputs of sensors, in particular, physiological sensors, and more particularly, sensors for electroencephalogram (EEG) and magnetoencephalogram (MEG) measurements.

### BACKGROUND OF THE INVENTION

Currently, most, if not all, commercially available devices for recording physiological signals are subject to line noise, the spurious electrical signals derived from the electrical supply to a sensor device, the noise signals often masking the electrical signals attributable to the physiological process of interest. Line noise appears at a frequency of 60 Hz in North America and 50 Hz in other regions throughout the world in accordance with the frequency of the local electrical current. Line noise may be conducted or radiated in origin. Examples of devices affected by line noise include, but are not limited to, devices and systems for recording EEG, MEG, electromyogram (EMG), electrocardiogram (EKG or ECG), ballistocardiogram (BKG), electrooculogram (EOG), electrodermalgram (EDG), electrodermal activity (EDA), or eyelid movement (ELM).

It is known in the art to minimise line noise from physiological signal measurements of interest simply by applying a notch filter, which essentially comprises of a combination of a steep low-pass filter and a high-pass filter. While effective, this type of signal filtration can distort the signal of, for example, an EEG in spectral proximity to the effective range of the notch filter. Consequently, high-frequency cortical oscillations occurring in the upper gamma band range (50-60 Hz) are compromised by such notch filtering solutions. In addition to such standard filtering approaches, other attempts to remove line noise from EEG data, for example, include spatial implementations of principal and independent components analysis and wavelet de-noising. While such approaches can be used effectively to minimize line noise offline, they are typically not applied under online, real-time recording conditions.

What is needed is a method for line-noise suppression that removes line noise from signals effectively but does not distort the remaining signals that represent the physiological signal of interest. Ideally, the line-noise suppression would enable the signal analysis to occur no later than a short time after the signals are collected, effectively, in “real-time” or “near real-time”.

### SUMMARY OF THE INVENTION

It is an object of the invention to provide a method and apparatus for acquiring physiological signals from a subject and removing line noise from the signals with minimal distortion of the signal or signals of interest. It is a further object of the invention to provide a method and apparatus that is operable in real-time or near real-time. Other objects will become evident on reading the detailed description of the invention. It will be understood that the scope of the invention is not limited to the embodiments described in the description but that the scope includes embodiments within the scope of the appended claims.

The present invention provides a method of overcoming the contamination of desired physicological signals with periodic, replicable signals, or noise, caused by inherent electrical charateristics of the electrical supply to electrical measuring devices. The method of the invention exploits the periodic and spectrally stationary nature of line noise, which is spectally constant at its frequency of origin, but may vary over time with respect to location-specificity and time-varying amplitude. The method can be advantageously implemented in computer software for easy calculation and display of calculated results for interpretation and use of the resulting relatively uncontaminated signals. The method can be applied in applications wherein measurements are made of physiological parameters of humans or any other animal, as appropriate.

In one aspect, the invention provides a method for processing data acquired from physiological sensors, comprising the steps of collecting raw sensor data in a file, said data representing at least one electrophysiological signal; selecting a time interval that is a whole-number multiple of the period of the waveform of said at least one signal; calculating an average value of the data for each of a series of consecutive time periods in the data file wherein said time period is a whole-number multiple of the time period of an artefact waveform; calculating a standard cross-correlation value for the calculated average from the sampling period according to the spectral peak and the raw data collected over the same time interval; and subtracting the average calculated according to the sampling period from the raw data in each time period.

In another aspect, the invention provides a method for acquiring and processing physiological signals acquired from a subject, comprising the steps of locating at least one sensor to acquire a least one physiological signal from a subject; acquiring a least one physiological signal from said at least one sensor; selecting a time interval that is a whole-number multiple of the period of the waveform of said at least one signal; transforming the at least one signal into raw data in a format suitable for data storage; storing the raw data at least one signal in at least one data storage means; and for each sensor, calculating an average value of the sensor output for each of a series of consecutive time periods in the data file wherein said time period is a whole-number multiple of the time period of an artefact waveform, providing a dynamic average value for the time periods; calculating a standard cross-correlation value for the calculated average for each sensor for each of the series of time periods and the raw data measured and stored over the same time interval; and subtracting the dynamic average from the raw data in each time period.

In a further aspect, the invention provides a method for processing data acquired from physiological sensors, comprising the steps of collecting raw sensor data in a file, said data representing at least one electrophysiological signal; identifying the spectral peak of an artefactual waveform in the at least on electrophysiological signal; calculating a sampling period according to the spectral peak; calculating an average value of the data for each of a series of consecutive time periods in the data file wherein said time period is a whole-number multiple of the sampling period of the artefact waveform; calculating a standard cross-correlation value for the calculated average from data for each of the series of consecutive time periods and the raw data collected in step from a sensor over the same time interval; and subtracting the average calculated for each of the series time period from the raw data in each time period.

Preferably, the method includes a step of determining the sampling period according to the time period during which the spectral peak exceeds a threshold. Preferably, the at least one physiological signal comprises of a continuous stream of measurable input. Preferably, the method includes the step of determining the shift delay at the maximum value in the cross-correlation function and timeshifting the artefact average correspondingly. Preferably the method includes the step of creating and displaying a corrected data set. Preferably, the method includes the step of storing the calculated data in a computer file. Preferably, the method includes displaying the raw, uncorrected data. Preferably the waveform of the electrophysiological signal is any one of sinusoidal, square or triangular in graphical shape. Preferably, the steps of the method are carried out in real-time or near-real time.

In a still further aspect, the invention provides apparatus for acquiring and processing physiological signals from a subject including at least one sensor for acquiring at least one signal and at least one microprocessor means for processing said at least one signal, said at least one microprocessor means including means for storing a whole number multiple of an artefact waveform for calculating the line-noise component of data derived from said at least one sensor.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 shows a flow diagram of the steps used in the method of acquiring and analyzing biosignals.

FIG. 2 shows screen displays of an example of electrophysiological signals acquired and analyzed according to the invention.

FIG. 3 shows screen displays of an example of EEG signals acquired and analyzed according to the invention.

### DETAILED DESCRIPTION OF THE INVENTION

A method in accordance with the present invention includes acquiring a real-time (or near real-time) signal or signals using an electrical sensor device or devices having a source or sources of electrical power, followed by the transforming the acquired signal(s) according to an algorithm of the invention. It will be understood that the method can be used for one or more sensors simultaneously or in sequence.

Characteristics of the real-time data acquisition step may include the following. Data acquisition may be a continuous stream of measurable impulses comprising the targeted physiological signal from a sensor located adjacent, or in proximity to, the subject. Data may be stored in raw (uncorrected) format. It may be displayed and stored in the modified (corrected) format. An underlying assumption for the use of the algorithm to analyze signals is that the line noise or other such external continuous periodic source is defined to mean “a continuously or episodically present repetitive waveform” such as, for example, any one of a sinusoid, square wave, or triangular wave, or any other continuously repetitive artefactual activity measured in the physiological parameter of interest, where “artefactual” is defined to mean any activity that is not the targeted signal of interest.

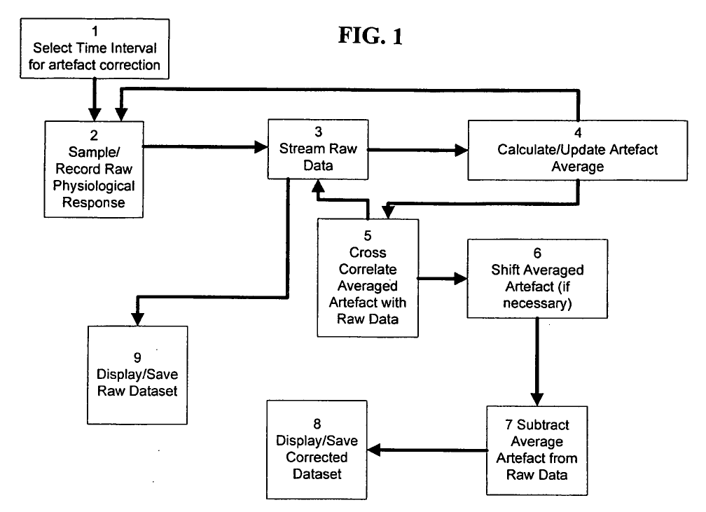

FIG. 1 shows steps in a method of the present invention, including, but not limited to, the following steps. It will be understood that the method preferably includes the additional steps of storing and/or displaying raw data representing acquired signals and corrected data but those steps need not to be practiced. The method includes the step of selecting a time interval that is a whole-number multiple of the period of the waveform of the target sensor signal 1. The sensor output is sampled, preferably continuously 2, resulting in a stream of raw data. The sensor output is recorded in a data file as it is sampled 3. An average value of the sensor output is calculated for each of a series of consecutive time periods in the data file 4, providing a dynamic average value for the time periods. A standard cross-correlation value is calculated from the calculated average from box 4 and the stored measured raw data 3 over the same time interval 5. Optionally, a shift delay at the maximum value in the cross-correlation function may be calculated and the artefact average is time-shifted correspondingly 6, if a shift is required. The dynamic average of the artefact is subtracted from the raw data in each time period 7. Preferably the method includes creating, displaying and storing a corrected data set 8 comprised of the results from calculating the average 4 concurrently with the raw data set 3. Preferably the raw, uncorrected data is displayed or stored 9.

The method of the invention may include real-time data acquisition using an electrical sensor device having a source of electrical power and the transformation of acquired data according to the algorithm of the invention.

The method of the invention may include repeating steps 4 to 7 in FIG. 1 for successive time periods based on the period of the artefact waveform. The successive time periods are preferably whole-number multiples of the period of the artefact waveform. Low multiples of the time period of the artefact are preferable as they provide for rapid correction and rapid updating of the average artefact used for subtraction purposes. According to the invention for step 1, for example, for a source of electrical current at 50 Hz powering the electrical sensor device, this would be 20 ms (or any whole number multiple of 20). For a 60 Hz artefact, this interval would be a whole number multiple of 16.666 ms (or any whole-number multiple). Alternatively, a common value of 500 ms could be used, which would provide an interval that is a whole number multiple of the period for either 50 or 60 Hz. The whole number may be hard-coded in hardware. Alternatively, it may be a user-controlled parameter in computer software. Alternatively, it may be dynamically adjusted to provide the maximum suppression of line (mains) based on a real-time spectral amplitude/power measure of the targeted artefact.

According to the invention, for steps in boxes 2-6 the physiological response is continuously sampled and an average signal is calculated for the physiological activity recorded from consecutive periods. The number of sampled periods used to generate the average may be fixed (e.g., 10 sampled periods) or it may be a user-determined value. The average calculated according to box 4 is dynamically updated so that as the number of sampled periods for the average is fulfilled, the first sample in a period is dropped and replaced by the next sampled period. Based on this procedure, activity that is time-locked and/or phase-locked to the spectral period of the artefact signal is maintained with amplitude equal to the average of the sampled epochs when in the average, while all other non-time-locked or phase-locked activity to the period of the artefact is diminished because of the absence of phase coherence.

An embodiment of the present invention includes that a dynamic average is continuously subtracted from the raw data (buffered or streamed in real-time) and sent to a corrected data set collected concurrently with the raw data set. This corrected data set maybe used for display purposes only. Alternatively, it may be saved concurrently with, or instead of, the raw data set.

According to an aspect of the invention, as shown in box 7 of FIG. 1, to avoid single point offsets of the actual data to the sampled average used for correction, a standard cross-correlation method based on the best correlation match for the sampled average and the raw data over the same time interval, is used to time-shift the sampled average to obtain the best correction. If there is no time-shift in the peak of the cross-correlation function, the average would be applied directly to the matching segment of buffered data. However, it there is a time-shift in the peak of the cross-correlation function, the average is shift by the number of data points equivalent to the time shift in the cross-correlation analysis. Once this time-shift is performed, the averaged artefact is subtracted from the raw data.

Alternate embodiments of the invention may include a spectral detection method that automatically identifies the spectral peak of the artefact in the physiological data and then calculates the appropriate sampling period to apply the correction. Alternatively, the method may include a whole-number multiple of the sampling period for use in the calculation. This embodiment could also include a threshold detection measure for spectral amplitude and for a minimum time period before the “repetitive” activity would be regarded as artefactual to avoid removing, for example, real EEG signals, such as alpha oscillations, for example. Similar corrections can also be applied offline.

Other embodiments of the invention may include correction of electrical signals from sensor devices for any repetitive electrical source producing a well characterized or deterministic artefact signature. Such examples would specifically include the artefact produced by trans-cranial magnetic stimulation or electrical/mechanical somatosensory stimulation, electrical pump noise associated with the delivery of coolant in MRI (magnetic resonance imaging) environments or other similar sources of artefact signal.

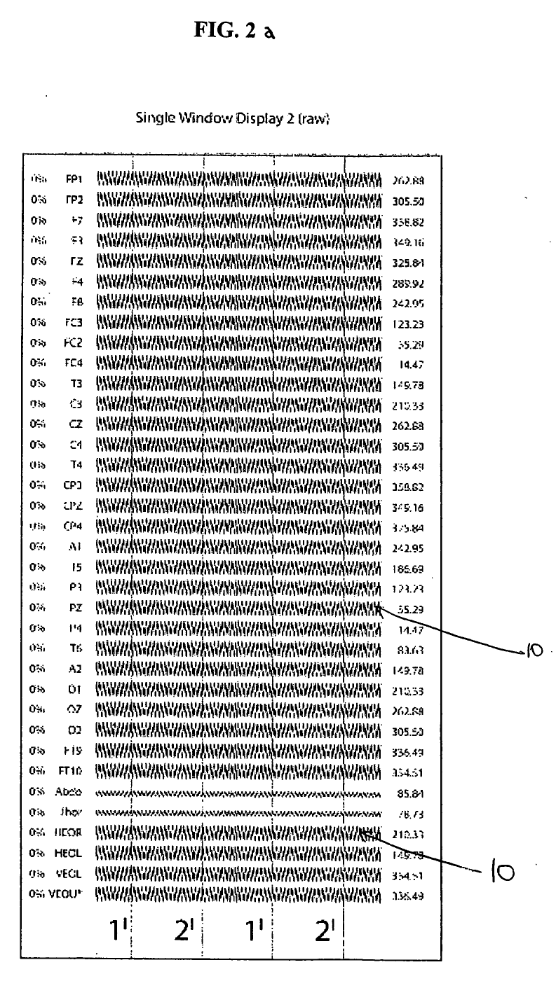

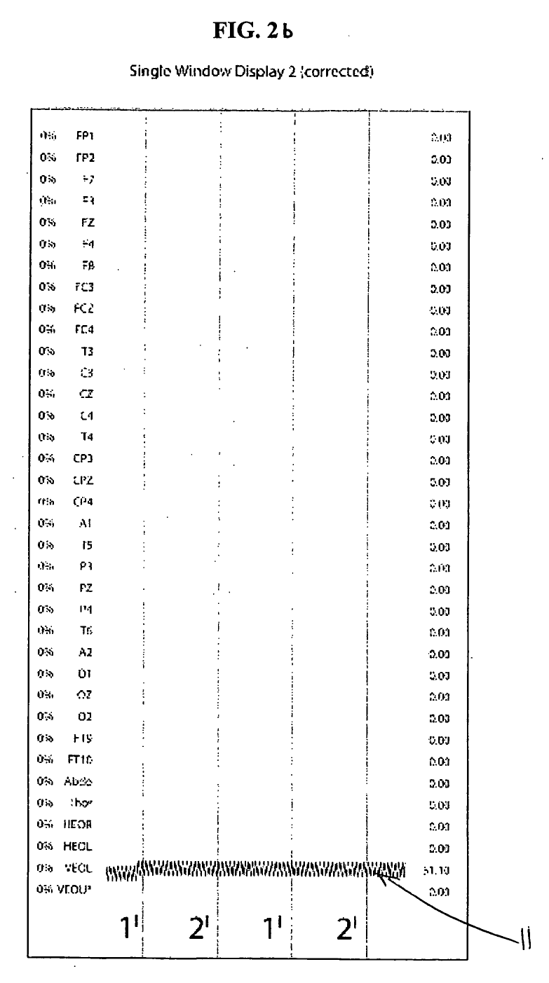

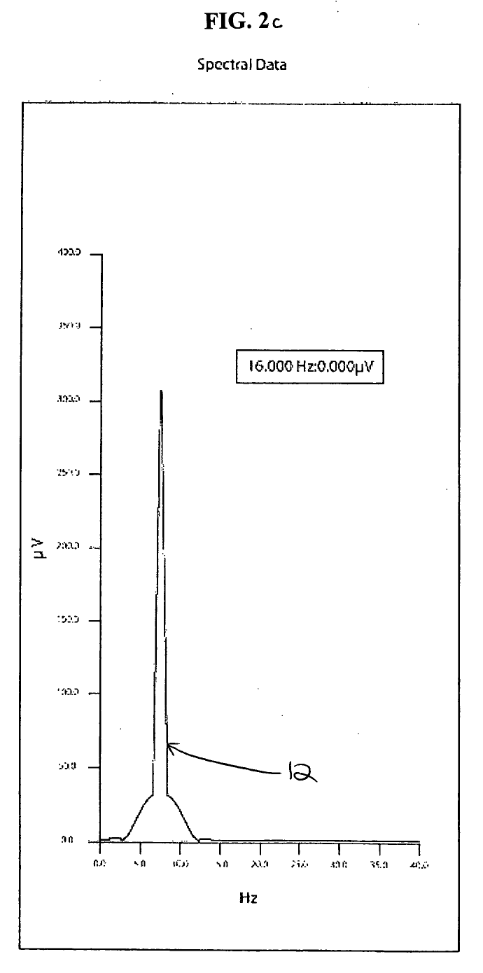

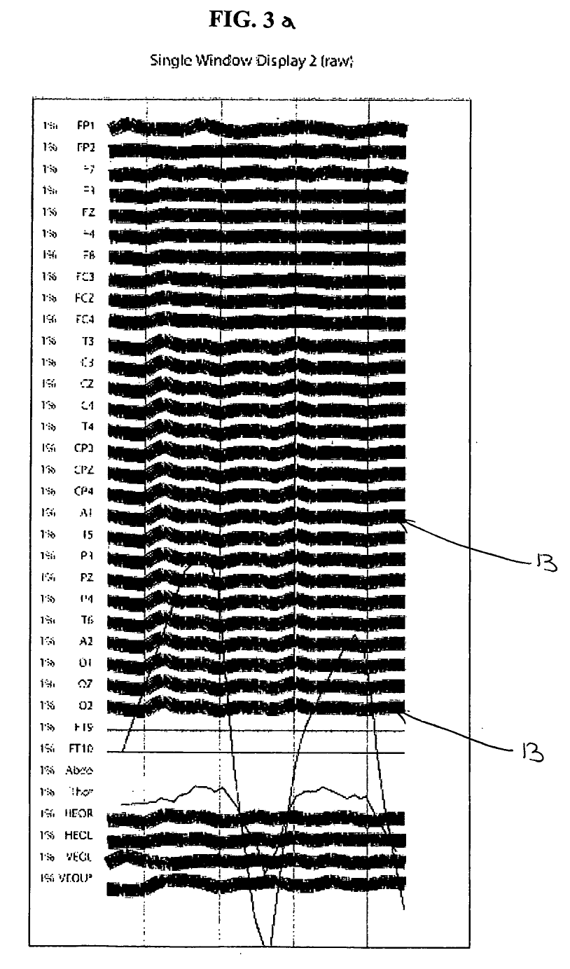

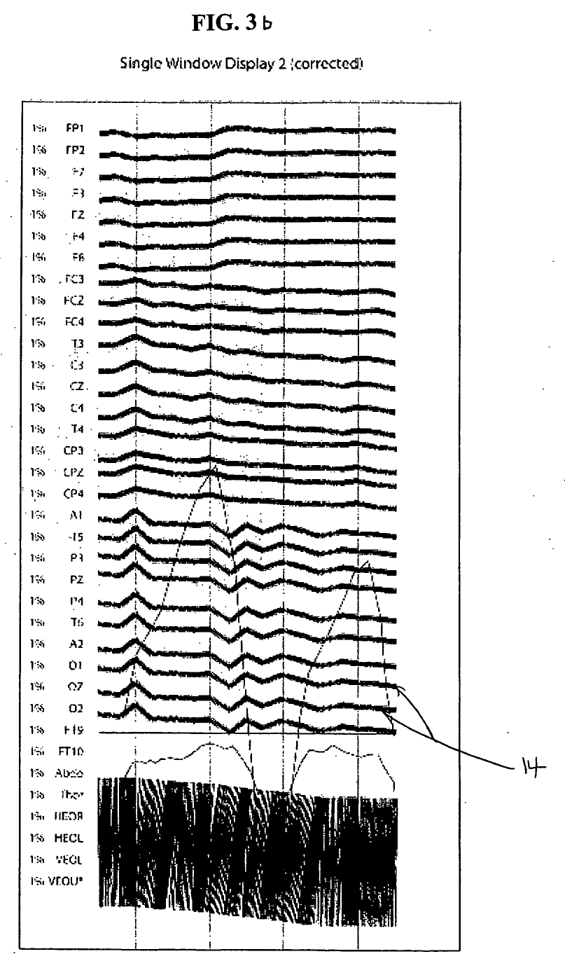

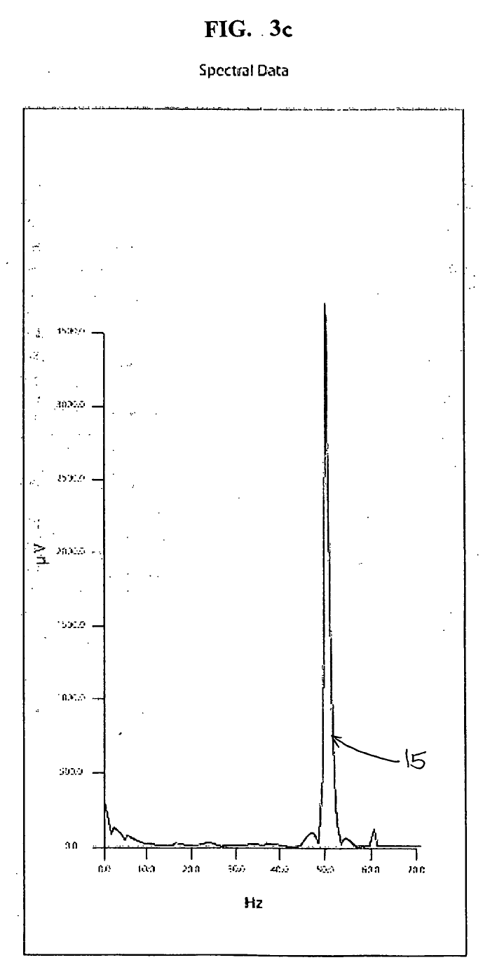

Illustrations of the adaptive noise removal for simulated and real data are shown in FIGS. 2 and 3.

The results for the simulated data are shown in FIGS. 2 and 3. The uncorrected data 10 is shown in FIG. 2A, corrected (except for one channel 11) shown in FIG. 2B, and the spectral peak 12 of the uncorrected versus corrected are shown in the graph FIG. 2C. With simulated data there is perfect correction, with no residual evidence of the continuously periodic waveform shown in FIG. 2A. The reduction in the simulated line noise is essentially infinite.

Correction of real data, in this case from an EEG recording, is shown in FIG. 3C. The raw data 13 is in FIG. 3A, the corrected data 14 in FIG. 3B, and the spectral comparison of the data 15 is shown in FIG. 3C. The reduction in spectral energy at 50 Hz line frequency is from over 4000 microvolts to less than 100 microvolts. The residual 50 Hz line frequency noise is approximately 1/800 the amplitude of the uncorrected data or nearly 60 dB of line (mains) suppression.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method for processing data acquired from physiological sensors, said method comprising:
a) collecting raw sensor data in a file, said data representing at least one electrophysiological signal;
b) selecting a time interval that is a whole-number multiple of the period of the waveform of said at least one signal;
c) calculating an average value of the data for each of a series of consecutive time periods in the data file wherein said time period is a whole-number multiple of the time period of an artefact waveform;
d) calculating a standard cross-correlation value for the calculated average from step c) and the raw data collected in step a) over the same time interval; and
e) subtracting the average calculated according to step c) from the raw data in each time period.

2. A method for acquiring and processing physiological signals acquired from a subject, said method comprising:
a) acquiring at least one physiological signal from at least one sensor on a subject;
b) selecting a time interval that is a whole-number multiple of the period of the waveform of said at least one signal;
c) transforming the at least one signal into raw data in a format suitable for data storage;
d) storing the raw data in at least one data storage means;
e) for each sensor, calculating an average value of an output of the sensor for each of a series of consecutive time periods in the data file wherein said time period is a whole-number multiple of the time period of an artefact waveform, providing a dynamic average value for the time periods;
f) calculating a standard cross-correlation value for the calculated average from step e) and the raw data measured in step c) over the same time interval; and
g) subtracting the dynamic average from the raw data in each time period.

3. A method for processing data acquired from physiological sensors, said comprising:
a) collecting raw sensor data in a file, said data representing at least one electrophysiological signal;
b) identifying the spectral peak of an artefactual waveform in the at least one electrophysiological signal;
c) calculating a sampling period according to the spectral peak;
d) calculating an average value of the data for each of a series of consecutive time periods in the data file wherein said time period is a whole-number multiple of the sampling period of the artefact waveform;
e) calculating a standard cross-correlation value for the calculated average from step d) and the raw data collected in step a) over the same time interval; and
f) subtracting the average calculated according to step d) from the raw data in each time period.

4. The method of claim 3 further comprising a step of:
determining the sampling period according to the time period during which the spectral peak exceeds a threshold.

5. The method of claim 1 wherein the at least one physiological signal comprises of a continuous stream of measurable input.

6. The method of claim 2 wherein the at least one physiological signal comprises of a continuous stream of measurable input.

7. The method of claim 3 wherein the at least one physiological signal comprises of a continuous stream of measurable input.

8. The method of claim 1 further comprising the step of determining the shift delay at the maximum value in the cross-correlation function and timeshifting the artefact average correspondingly.

9. The method of claim 2 further comprising the step of determining the shift delay at the maximum value in the cross-correlation function and timeshifting the artefact average correspondingly.

10. The method of claim 3 further comprising the step of determining the shift delay at the maximum value in the cross-correlation function and timeshifting the artefact average correspondingly.

11. The method of claim 4 further comprising the step of determining the shift delay at the maximum value in the cross-correlation function and timeshifting the artefact average correspondingly.

12. The method of claim 5 further comprising the step of determining the shift delay at the maximum value in the cross-correlation function and timeshifting the artefact average correspondingly.

13. The methods of claims 1-12 further comprising the step of creating and displaying a corrected data set.

14. The method of claim 1 further comprising the step of storing the calculated data in a computer file.

15. The method of claim 3 further comprising the step of storing the calculated data in a computer file.

16. The method of claim 4 further comprising the step of storing the calculated data in a computer file.

17. The method of claim 1 further comprising displaying the raw, uncorrected data.

18. The method of claim 2 further comprising displaying the raw, uncorrected data.

19. The method of claim 3 further comprising displaying the raw, uncorrected data.

20. The method of claim 1 wherein said waveform is any one of sinusoidal, square or triangular in graphical shape.

21. The method of claim 2 wherein said waveform is any one of sinusoidal, square or triangular in graphical shape.

22. The method of claim 3 wherein said waveform is any one of sinusoidal, square or triangular in graphical shape.

23. The method of claim 1 wherein the steps of the method are carried out in real-time or near-real time.

24. The method of claim 2 wherein the steps of the method are carried out in real-time or near-real time.

25. The method of claim 3 wherein the steps of the method are carried out in real-time or near-real time.

26. An apparatus for acquiring and processing physiological signals from a subject including at least one sensor for acquiring at least one signal and at least one microprocessor means for processing said at least one signal, said at least one microprocessor means including means for storing a whole number multiple of an artefact waveform for calculating the line-noise component of data derived from said at least one sensor.
